(19) United States
(12) Patent Application Publication
(10) Pub. No.: US 2022/0395335 A1
(43) Pub. Date: Dec. 15, 2022

(54) ENDOSCOPIC MAGNETIC GUIDANCE SYSTEM AND METHODS

(71) Applicant: Ethicon, Inc., Somerville, NJ (US)

(72) Inventor: Matthew Kuhn, Houston, TX (US)

(21) Appl. No.: 17/828,432

(22) Filed: May 31, 2022

Related U.S. Application Data
(60) Provisional application No. 63/210,329, filed on Jun. 14, 2021.

(51) Int. Cl. A61B 34/20

(52) U.S. Cl. CPC A61B 34/20; A61B 2034/2051

(57) ABSTRACT

The present disclosure relates to systems and methods to magnetically influence the position, orientation, movement, and/or activation of a medical device within an operating region within the body using a magnetic guidance system introduced into a target location of a body. More specifically, the present disclosure relates to magnetic navigation systems for applying a magnetic field to an operating region within the body, the system comprising a first magnet assembly configured for introduction into a target location of a body and a second magnet assembly disposed opposite the first magnet assembly, wherein the first and second magnet assemblies are configured for applying a magnetic field to an operating region between the magnet assemblies.

### CROSS-REFERENCE TO RELATED APPLICATIONS

This application claims priority to and the benefit of U.S. Patent Application No. 63/210,329 filed Jun. 14, 2021, which is hereby incorporated by reference in their entirety.

### BACKGROUND

Significant progress has been made in automating the navigation of medical devices in the body. Remote navigation systems allow a physician to remotely orient the distal end of a medical device in the body. In recent years, magnetically navigable and controllable catheters have been used. These catheters have allowed more aspects of ablation and mapping procedures to be automated for improved accuracy and efficiency. They also provide the benefit of lowering radiation exposure at least for the treating physicians by enabling catheter control from a remote location away from the patient. These devices typically house a position sensor and a sufficient volume of magnetic or magnetizable material to enable suitable magnetic response to an external magnetic guidance system.

A magnetic guidance system 100, as shown in FIG. 1 for example, typically utilizes two large permanent magnets 104 and 106 mounted on either side of the patient table 102 that generate magnetic navigation fields. The magnets allow for controlling the position and orientation of magnet devices such as catheters introduced into the body. As seen in FIG. 1, these systems are large, space encumbering, comprise many complex electromechanical systems, and can be extremely costly. Furthermore, the 6-degrees of freedom (“DOR”) control of magnetic devices afforded by many magnetic guidance systems, which enables total robotic guidance of device introduction, navigation, orientation, and activation within the body, may not be required for all use-cases. For example, navigation of an electrophysiology catheter into the heart may be performed manually, after which there is only a need for improved maintenance of contact force or more controlled mapping.

Improvements are needed.

### SUMMARY

Disclosed herein are magnetic navigation systems and methods for applying a magnetic field to an operating region within the body. One general aspect includes a magnetic navigation system for applying a magnetic field to an operating region within the body. The magnetic navigation system also includes a first magnet assembly configured for introduction into a target location of a body; and a second magnet assembly disposed opposite the first magnet assembly, where the first and second magnet assemblies are configured for applying a magnetic field to an operating region between the magnet assemblies. Other embodiments of this aspect include corresponding computer systems, apparatus, and computer programs recorded on one or more computer storage devices, each configured to perform the actions of the methods.

One general aspect includes a system for generating a magnetic field. Other embodiments of this aspect include corresponding computer systems, apparatus, and computer programs recorded on one or more computer storage devices, each configured to perform the actions of the methods.

One general aspect includes a magnetic field source for generating a magnetic field. Other embodiments of this aspect include corresponding computer systems, apparatus, and computer programs recorded on one or more computer storage devices, each configured to perform the actions of the methods.

One general aspect includes a magnetic navigation system for applying a magnetic field to an operating region within the body. The magnetic navigation system also includes a first magnet assembly configured for introduction into a target location of a body; a second magnet assembly disposed opposite the first magnet assembly external to the body, where the first and second magnet assemblies are configured for applying a magnetic field to an operating region between the magnet assemblies; and at least one magnet assembly support for one or more of supporting or moving at least one magnet assembly to change the direction of magnetic field applied to the operating region. Other embodiments of this aspect include corresponding computer systems, apparatus, and computer programs recorded on one or more computer storage devices, each configured to perform the actions of the methods.

One general aspect includes a system for orienting a medical device within an operating region within the body. The system also includes a first magnet assembly configured for introduction into a target location of a body; a second magnet assembly disposed opposite the first magnet assembly, where the first and second magnet assemblies are configured for applying a magnetic field to an operating region between the first and second magnet assemblies; and a magnetically-responsive medical device configured to magnetically interact with the first and second magnet assemblies. Other embodiments of this aspect include corresponding computer systems, apparatus, and computer programs recorded on one or more computer storage devices, each configured to perform the actions of the methods.

One general aspect includes a magnetic navigation system for applying a magnetic field to an operating region within the body. The magnetic navigation system also includes an elongate body configured to be delivered within a body lumen, the elongate body including a distal portion and a proximal portion, where the distal portion may include a first magnet assembly; a second magnet assembly disposed opposite the first magnet assembly external to the body, where the first and second magnet assemblies are configured for applying a magnetic field to an operating region between the first and second magnet assemblies. Other embodiments of this aspect include corresponding computer systems, apparatus, and computer programs recorded on one or more computer storage devices, each configured to perform the actions of the methods.

### BRIEF DESCRIPTION OF THE DRAWINGS

The following drawings show generally, by way of example, but not by way of limitation, various examples discussed in the present disclosure. In the drawings:

FIG. 1 depicts an exemplary robotic magnetic guidance system in accordance with the prior art.

FIG. 2 depicts an exemplary magnetic catheter introduced into a heart via a magnetic guidance system.

FIG. 3 depicts an exemplary catheter body.

FIG. 4 depicts an exemplary catheter tip.

FIG. 5 depicts an exemplary catheter tip.

FIG. 6A depicts an exemplary magnetic catheter introduced into a heart.

FIG. 6B depicts an exemplary magnetic guidance system introduced into an esophagus.

FIG. 6C depicts exemplary movement of the magnetic catheter with the magnetic guidance system.

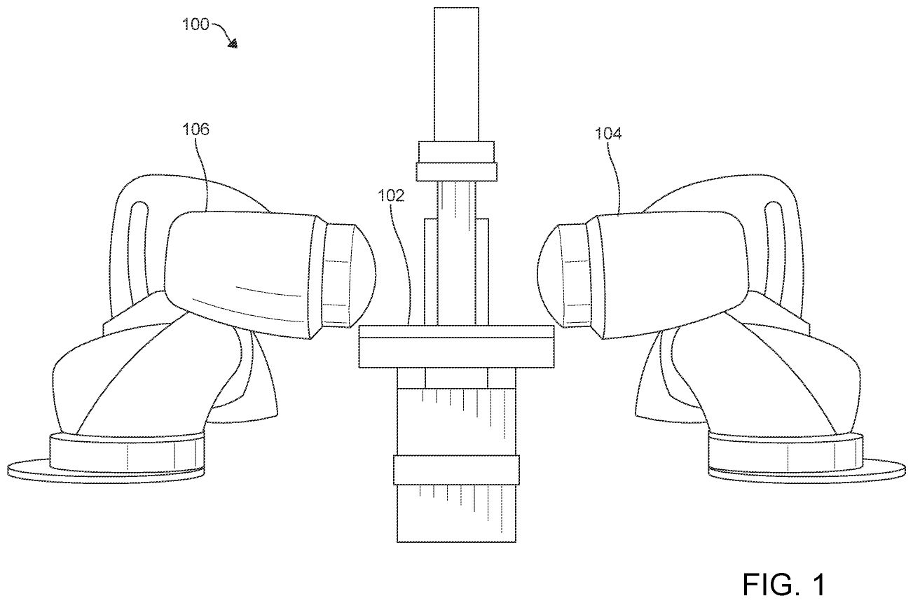

### DETAILED DESCRIPTION

For certain cardiac mapping and ablation procedures the quality of the mapping and/or ablation depends upon the quality of the contact between the electrode and the cardiac tissue. It is difficult to maintain the desired contact with the moving surface of the heart during the entire cardiac cycle. Typically, relatively stiff medical devices are urged against the surface of the heart with a certain amount of force in an attempt to maintain contact during the entire cardiac cycle.

Early magnetic navigation techniques involved the use of superconducting magnets. While these techniques were, and remain, highly effective, advances in permanent magnetic materials and in the design of permanent magnets, have made it possible to use permanent magnets for magnetic navigation. While the magnetic fields created by superconducting magnets can be readily changing the currents in the superconducting electromagnetic coils, in order to change the magnetic field created by permanent magnets for navigation, it is generally necessary to change the position and/or orientation of the magnetic field applied by permanent magnet by accurately controlling the position and/or orientation of the permanent magnet.

The present disclosure relates to a system for magnetically navigating a medical device in an operating region within the body of a patient, in which a catheter-based device with an array of tunable/adjustable/translatable/rotatable permanent magnets is endoscopically positioned posteriorly adjacent the heart and a corresponding magnet unit is secured externally at a position anteriorly adjacent the heart (i.e. applied to the patient's chest), wherein the system generates a dynamically controllable magnetic field within the operating region of the heart. The magnetic field can then be leveraged by a magnetically-responsive medical device such as a catheter with an orthogonal array of coils that are selectively exercisable to generate precise magnetic moments to cause the device to change orientation within the operating region.

A plurality of field generators can be disposed and energized to generate converging (co-facing) toroidal magnetic fields. Disposition is either coaxial along a common bore axis in the case of two field generators, or at equivalent angles to shared axes (orthogonal disposition) when three or more field generators are applied. Magnetic fields can be generated and focused into higher flux densities toward a convergence plane disposed midway between field generators.

According to another aspect of the disclosure the system includes a support for mounting and/or changing the position and orientation of the external magnet assembly to change the direction of magnetic field applied to the operating region. The support is preferably capable of pivoting the magnet about a first axis that rotates about a second axis perpendicular to the first axis, and translating the magnet, preferably parallel to the second axis.

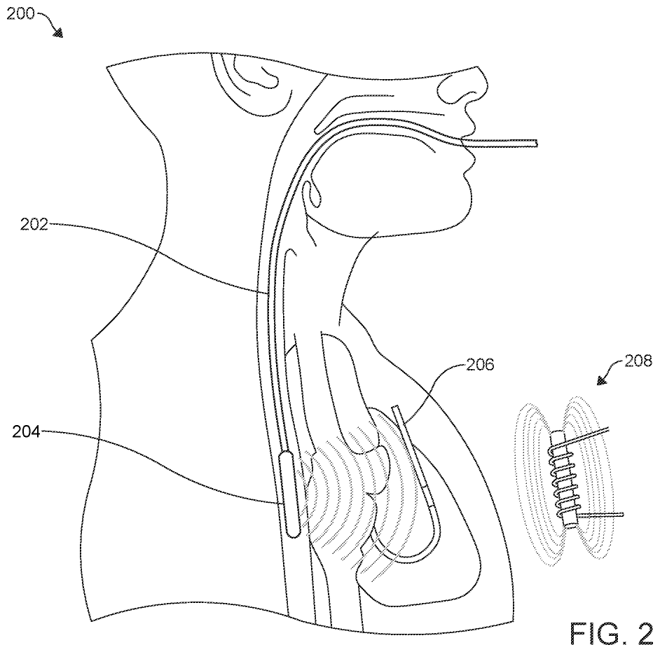

FIG. 2 depicts an overview of the system. FIG. 2 shows a human chest cavity 200 in cut away view. An endo-esophageal magnetic guidance device 204 is placed into a patient's esophagus with a cable 202 for communication with the endo-esophageal magnetic guidance device. A magnetic catheter 206 is placed in the heart. The corresponding magnetic guidance system 208 is placed on the chest. The guidance system interfaces with the endo-esophageal magnetic guidance device to help control the position and orientation of the magnetic catheter 206.

In one embodiment, the present disclosure relates to a magnetic navigation system, and in particular to a system including magnet units comprising a permanent magnet, and a support for controlling the position and orientation of a permanent magnet. The system is adapted for magnetically navigating a medical device in an operating region within the body of a patient. Generally, the system comprises a magnet having a front field projecting from the front of the magnet sufficient to project a magnetic field into the operating region in the patient. The magnet is mounted for movement between a navigation position in which the magnet is located adjacent to the patient with the front of the magnetic generally facing the operating region, and an imaging position in which the magnet is spaced from the patient and the front generally faces away from the operating region.

In one embodiment, the method and apparatus of the present disclosure facilitate the placement of the distal end of a medical device, such as a catheter or micro-catheter, against a target location on a three-dimensional curved surface within a subject body. The method may further provide the feature of determining an external magnetic field to be applied to the medical device for providing a desired estimated contact force against the tissue surface within the subject body. These and other features and advantages will be in part apparent, and in part pointed out hereinafter.

In one embodiment, a method for establishing and estimating the contact force of the tip of a medical device against a tissue surface within a subject body is provided in accordance with the principles of the present disclosure. In one embodiment, the method provides for estimating the contact force of a medical device with a tissue surface such as the heart, through the suitable estimation of the torque applied to the medical device via a magnetic field. While this embodiment is operable with magnetically navigable medical devices, other embodiments of a method in accordance with the present disclosure may be used with medical devices that are guided without magnetic navigation but instead use other control methods for remote navigation such as mechanical actuation, electrostrictive actuation, or hydraulic actuation.

By monitoring the force from the sensor, the remote navigation system may be operated to maintain a satisfactory contact force, either by determining a condition (orientation and position) in which the sensed force is maintained between predetermined minimums and maximums, throughout the entire cardiac cycle, or by dynamically changing the condition (position and orientation) to maintain the sensed force between predetermined minimums and maximums.

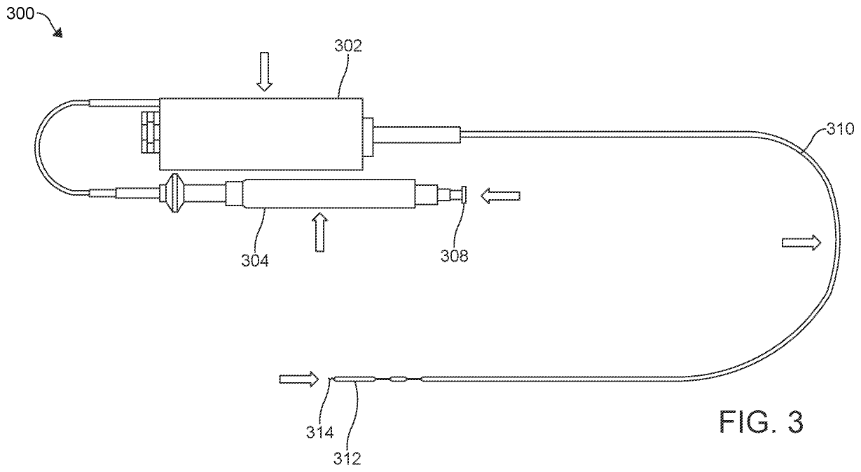

FIGS. 3-5 depict an exemplary electrophysiology catheter that is adapted for ablation, mapping, injection, and directional control. In one embodiment, the catheter has a catheter body comprising a handle 302 and a needle deployment/retraction mechanism 304, an intermediate section 310, and a tip section 312 having a tip electrode configured with an omnidirectional distal end and a concentric needle port. The omnidirectional distal end of the tip electrode improves maneuverability and angulation. A dome configuration enables a wide range of tissue contact angles. The concentric needle port provides optimal tissue injection success.

FIG. 3 depicts details of the catheter 300. A needle control handle 302 is attached at the proximal end of a deployment and retraction mechanism 304 for a spring loaded injection needle 314. This mechanism 304 is connected to a fluid connection 308. The catheter intermediate section or shaft 310 connects the needle control handle 302 distally with the catheter tip section 312 and injection needle 314.

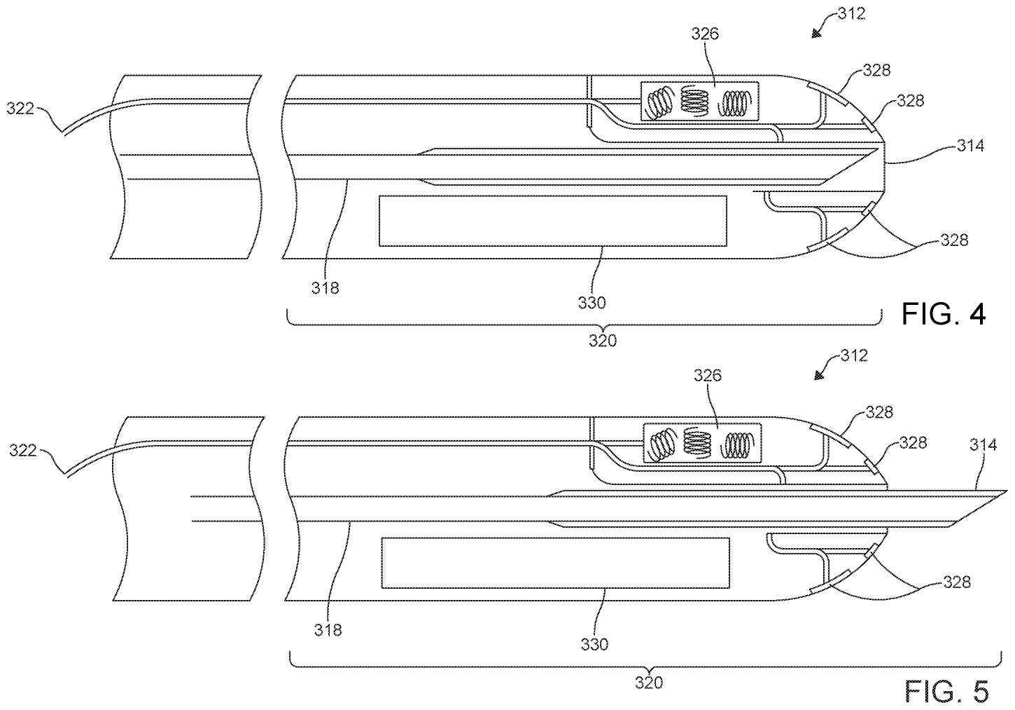

FIGS. 4 and 5 show details of the catheter tip 312. The injection needle 314 is retracted in FIG. 3B and deployed in FIG. 3C. In one embodiment, the tip section 320 houses a position sensor 326 arranged in an integrated configuration, wherein the configuration facilitates a path 318 in the tip section 320 for a component, including an injection needle 314, to extend through the tip section 320 for extension and retraction with reduced stress and friction. The integrated configuration is an efficient use of space that allows the tip section 320 to carry both the position sensor 326 for determining location and orientation of the tip section and the contact sensor or sensors 328 (four are depicted in this embodiment). A magnetic element 330 is also included to allow the magnetic guidance 204 206 system to control the positioning and orientation of the tip section 320. In addition, wiring 322 from the sensors connects to the needle control handle 302. The path 318 defined by the integrated position sensor 326 through the tip section 320 may be generally linear or nonlinear depending on the structure design of the position sensor. For the injection needle 314, the path 318 connects with the concentric needle port whether the path is on axis or off axis with the tip section 320. The catheter also includes a very soft and flexible intermediate section or shaft 310. The catheter tip section 320 may also comprise contact sensors 328 which confirm when the tip section 320 has contacted a body or surface, for instance, the endocardial surface. The injection needle 314 may be off axis or on axis relative to the contact sensor 328

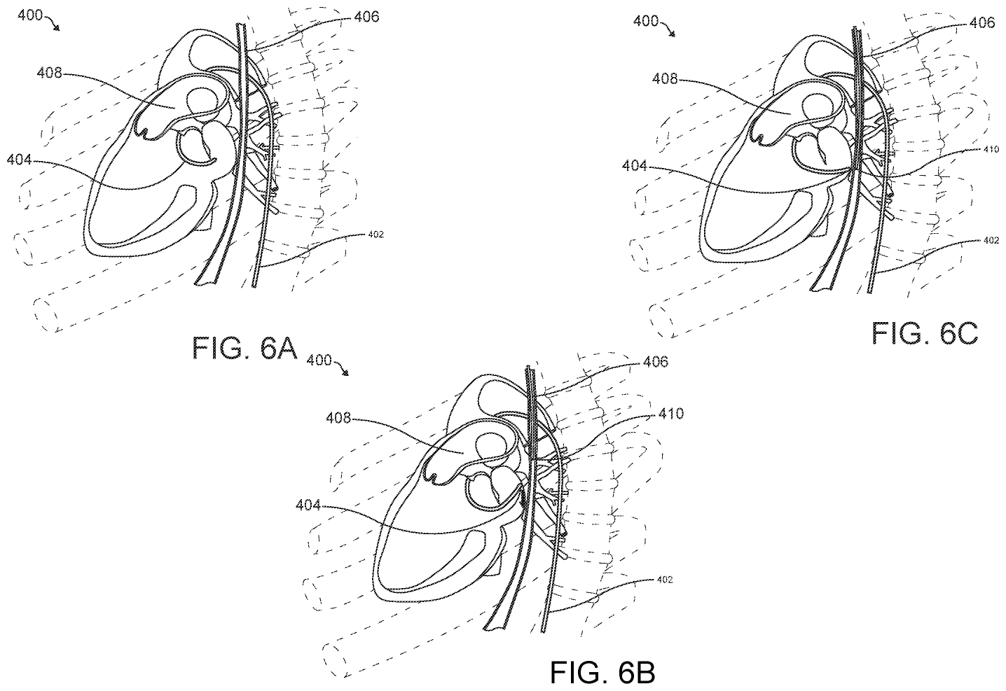

FIGS. 6A-6C depict the catheter in operation. FIG. 6A shows the human heart in 3D section with a catheter 400. The esophagus 406 and the heart 408 are shown most clearly with the skeleton transparent. The magnetic catheter 404 is introduced into a chamber of the heart. The magnetic catheter 404 communicates by the shaft 402 of a catheter. FIG. 6B depicts the introduction of the trans-esophageal catheter 410 with a magnetic element into the esophagus 406. The magnetic element 330 in the magnetic catheter 320 is drawn to the magnetic guidance device 410. The black arrow downward indicates the direction the magnetic catheter 404 and the esophageal magnetic guidance device 410 will travel. FIG. 6C indicates the new location of the magnetic catheter 404 and the magnetic guidance device 410.

Example magnetic navigation systems for applying a magnetic field to an operating region within the body are disclosed herein. Magnetic navigation systems may be endoscopic, minimally-invasive, intraluminal and/or transesophageal. An example system may comprise a first magnet assembly configured for introduction into a bodily passageway (e.g., a body cavity, a vessel, etc., such as an esophagus) and a second magnet assembly disposed opposite the first magnet assembly. The first and second magnet assemblies may be configured for applying a magnetic field to an operating region between the magnet assemblies. As an example, a first device may be placed into the esophagus and a second device applied to the outer surface of the chest, whereby both devices create a magnetic field between them that would include the patient's heart.

In an example embodiment. a magnetic navigation system for applying a magnetic field to an operating region within the body may comprise a first magnet assembly configured for introduction into a target location of a body and a second magnet assembly disposed opposite the first magnet assembly. The first and second magnet assemblies may each comprise at least one magnet configured for applying a magnetic field to an operating region between the magnet assemblies.

A magnetic navigation system for applying a magnetic field to an operating region within the body may comprise a first magnet assembly configured for introduction into a target location of a body and a second magnet assembly disposed opposite the first magnet assembly external to the body. The first and second magnet assemblies may each comprise at least one magnet configured for applying a magnetic field to an operating region between the magnet assemblies.

A magnetic navigation system for applying a magnetic field to an operating region within the body may comprise a first magnet assembly configured for introduction into a target location of a body and a second magnet assembly disposed opposite the first magnet assembly external to the body. The first and second magnet assemblies may be configured for applying a magnetic field to an operating region between the magnet assemblies. The system may further comprise at least one magnet assembly support for supporting at least one magnet assembly.

A magnetic navigation system for applying a magnetic field to an operating region within the body may comprise a first magnet assembly configured for introduction into a target location of a body and a second magnet assembly disposed opposite the first magnet assembly external to the body. The first and second magnet assemblies may be configured for applying a magnetic field to an operating region between the magnet assemblies. The system may further comprise at least one magnet assembly support for supporting and moving at least one magnet assembly to change the direction of magnetic field applied to the operating region.

A system for orienting a medical device within an operating region within the body may comprise a first magnet assembly configured for introduction into a target location of a body and a second magnet assembly disposed opposite the first magnet assembly. The first and second magnet assemblies may be configured for applying a magnetic field to an operating region between the first and second magnet assemblies. The system may further comprise a magnetically-responsive medical device.

A magnetic navigation system for applying a magnetic field to an operating region within the body may comprise an elongate body configured to be delivered within a body lumen, the elongate body including a distal portion and a proximal portion. The distal portion may comprise a first magnet assembly. The system may further comprise a second magnet assembly disposed opposite the first magnet assembly external to the body. The first and second magnet assemblies may be configured for applying a magnetic field to an operating region between the first and second magnet assemblies.

The magnet assemblies may include a positioner for rotating the magnet assemblies about a first axis and pivoting the magnet assemblies about a second axis.

The first and second magnet assemblies may comprise at least one permanent magnet. The magnetic field may be applied with a single permanent magnet, a pair of permanent magnets (for example in a gapped toroid arrangement), a single electromagnetic coil, two or more electromagnetic coils, or a combination thereof.

The first and second magnet assemblies may comprise a compound permanent magnet comprising a plurality of segments of magnetic material.

The system may further comprise a positioner for rotating the magnet about a first axis and pivoting the magnet about a second axis.

The magnet assemblies may be movably mounted on a support for movement about the body, and the magnet assemblies being positioned to project a magnetic field in a direction to orient a magnetically responsive medical device in a selected direction.

The system may further comprise rotating and pivoting each magnet assembly to maintain the magnetic field direction projected by the magnet assemblies in relation to the magnetically-responsive medical device as the magnet assemblies move on the support about an operating region of the body to maintain the magnetically-responsive medical device in a selected orientation as the magnet assemblies move on the support.

The positions of the magnet assemblies may be adjusted as the magnet assemblies move to change the direction of the magnetic field applied by the magnet assemblies to maintain the device in substantially the selected direction despite changes in the distance between the magnet assemblies and the operating region.

The system may further comprise a system controller to control a movement of a magnetically-responsive medical device. The controller may control the positioners of each of the magnet assemblies to change the positions of the magnet assemblies in response to a user-input to apply the magnetic field in the operating region to cause the magnetically responsive device to orient substantially in the selected direction. The controller may control the positioners of each of the magnet assemblies to change the positions of the magnet assemblies as the magnet assemblies move in order to maintain the magnetic field direction. The controller may control the positioners in response to movement of the magnet assemblies, to apply a field whose direction may be determined based upon a user-selected direction and a strength of the field in the operation region.

The first axes of the magnet assemblies may be parallel. Each of the magnet assemblies may also comprise a positioner for controlling the position of the magnet, e.g. for rotating the magnet about a first axis and pivoting the magnet about a second axis, to selectively change the magnetic field applied by each magnet to the operating region in a body on the subject support. In a preferred embodiment, the magnet assemblies, and thus their respective magnets, are mounted on opposite sides of the operating region. The first axis of rotation of each magnet preferably extends through the respective magnet and the operating region, and preferably the first axes of rotation of the magnets are co-linear. The second axes of pivoting of each magnet is preferably perpendicular to the first axis, and also rotates about the first axis.

The first axes of the magnet assemblies may be collinear and extend through the operating region.

The magnet assemblies may each comprise a compound permanent magnet comprising a plurality of segments of magnetic material with differing magnetization directions so that relatively small rotations or pivots change the magnetic field projected by the magnet at a specific point.

The magnet assemblies may be translatable along a first axis that extends radially outwardly from the center of the operating region, may be pivotable about a second axis, generally perpendicular to the first axis that extends through the center of mass of the magnet, and may be rotatable about the first axis.

The system may further comprise changing the magnetic moment of the magnetically-responsive medical device by selectively changing (i.e., via manual manipulation by a user, or a controller) a physical condition of at least one magnet element in the magnetically-responsive medical device to change the orientation of the device with respect to the applied magnetic field.

The system may further comprise applying electrical energy to create temporary magnetic moments in one or more coils in the magnetically-responsive medical device to change the orientation of the magnetically-responsive medical device with respect to the static magnetic field, and orient the magnetically-responsive medical device in a selected direction within the operating region.

A system for generating a magnetic field may comprise at least three magnetic field sources operable to radiate at least three magnetic fields into the anatomical body. Each magnetic field may have a moment different from each moment of each of the other two magnetic fields relative to a fixed point in space.

A magnetic field source for generating a magnetic field may comprise a first coil corresponding to a first magnetic pole and a second coil corresponding to a second magnetic pole. The first magnetic pole may be moveable with respect to said second magnetic pole.

### EXAMPLES

Example 1: A catheter comprising: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a magnetic element disposed in the tip section; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 2: The catheter of example 1, further comprising one or more position sensors disposed in or adjacent the tip section and configured to determine a positon of the tip section.

Example 3: The catheter of any one of examples 1-2, further comprising one or more position sensors disposed in or adjacent the tip section and configured to determine an orientation of the tip section.

Example 4: The catheter of any one of examples 1-3, further comprising one or more contact sensors disposed in or adjacent the tip section and configured to determine contact of the tip section to a surface.

Example 5: The catheter of any one of examples 1-4, wherein the distal end of the tip section has an omnidirectional configuration.

Example 6: The catheter of any one of examples 1-5, wherein the distal end of the tip section has a dome shape.

Example 7: The catheter of any one of examples 1-6, wherein a needle port is disposed on the distal end of the tip section and is configured to allow the needle to extend outside the distal end of the tip section.

Example 8: The catheter of any one of examples 1-7, wherein the needle port is disposed concentrically to the distal end of the tip section.

Example 9: The catheter of any one of examples 1-8, the catheter body comprises an intermediate section disposed between the tip section and the needle control handle.

Example 10: The catheter of any one of examples 1-9, wherein the intermediate section comprises a flexible tubing reinforced with a spring coil.

Example 11: An endo-esophageal magnetic navigation system comprising: a magnetic guidance device disposed external to a body of a patient; and a magnetic catheter further comprising: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a magnetic element disposed in the tip section, the magnetic element configured to magnetically interact with the magnetic guidance device to provide control over a position and orientation of at least the tip section; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 12: The endo-esophageal magnetic navigation system of any one of examples 1-11, further comprising one or more position sensors disposed in or adjacent the tip section and configured to determine a positon of the tip section.

Example 13: The endo-esophageal magnetic navigation system of any one of examples 1-12, further comprising one or more position sensors disposed in or adjacent the tip section and configured to determine an orientation of the tip section.

Example 14: The endo-esophageal magnetic navigation system of any one of examples 1-13, further comprising one or more contact sensors disposed in or adjacent the tip section and configured to determine contact of the tip section to a surface.

Example 15: The endo-esophageal magnetic navigation system of any one of examples 1-14, wherein the distal end of the tip section has an omnidirectional configuration.

Example 16: The endo-esophageal magnetic navigation system of any one of examples 1-15, wherein the distal end of the tip section has a dome shape.

Example 17: The endo-esophageal magnetic navigation system of any one of examples 1-16, wherein a needle port is disposed on the distal end of the tip section and is configured to allow the needle to extend outside the distal end of the tip section.

Example 18: The endo-esophageal magnetic navigation system of any one of examples 1-17, wherein the needle port is disposed concentrically to the distal end of the tip section.

Example 19: The endo-esophageal magnetic navigation system of any one of examples 1-18, the catheter body comprises an intermediate section disposed between the tip section and the needle control handle.

Example 20: The endo-esophageal magnetic navigation system of any one of examples 1-19, wherein the intermediate section comprises a flexible tubing reinforced with a spring coil.

Example 21: A method comprising: disposing a magnetic medical device in a body of a patient; navigating, using a magnetic guidance device disposed external to a body of a patient, the magnetic medical device to a target location in the body of the patient; determining, based on the magnetic interaction between the magnetic medical device and the magnetic guidance device, a torque or force applied to the medical device; determining, based on the torque or force applied to the medical device, a contact force between the magnetic medical device and a surface of tissue.

Example 22: The method of example 21, wherein the magnetic medical device comprises a magnetic catheter.

Example 23: The method of any one of examples 21-22, wherein the magnetic catheter further comprises: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a magnetic element disposed in the tip section, the magnetic element configured to magnetically interact with the magnetic guidance device to provide control over a position and orientation of at least the tip section; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 24: A method comprising: disposing a medical device in a body of a patient; navigating the magnetic medical device to a target location in the body of the patient; determining a navigation force applied to the medical device; and determining, based on the navigation force, a contact force between the magnetic medical device and a surface of tissue.

Example 25: The method of any one of examples 21-24, wherein the navigation force is applied using one or more of magnetic actuation, mechanical actuation, electrostrictive actuation, or hydraulic actuation.

Example 26: The method of any one of examples 21-25, wherein the magnetic medical device comprises a magnetic catheter.

Example 27: The method of any one of examples 21-26, wherein the magnetic catheter further comprises: a catheter body comprising a flexible tubing having a proximal end and a distal end and at least one lumen disposed through at least a portion of the flexible tubing; a tip section disposed adjacent the distal end of the catheter body, the tip section comprising a needle passage extending between a proximal end and a distal end, wherein the needle passage has a proximal region having a proximal diameter and a distal region having a distal diameter less than the proximal diameter; a magnetic element disposed in the tip section, the magnetic element configured to magnetically interact with the magnetic guidance device to provide control over a position and orientation of at least the tip section; a needle control handle disposed adjacent the proximal end of the catheter body; an injection needle disposed in at least a portion of the needle passage of the tip section, at least a portion of the catheter body, and at least a portion of the needle control handle, the injection needle having a proximal end coupled to the needle control handle and a distal end disposed within the needle passage, wherein the injection needle is longitudinally slidable so that the distal end of the injection needle extends beyond the distal end of the tip section upon suitable manipulation of the needle control handle; and a needle stop disposed on a portion of the injection needle, wherein the needle stop has a distal end that is sized to prevent passage of the portion of the injection needle on which the needle stop is mounted from passing into the distal region of the needle passage thereby limiting the distance that the injection needle is capable of extending beyond the distal end of the tip section.

Example 28: A magnetic navigation system for applying a magnetic field to an operating region within the body, the system comprising: a first magnet assembly configured for introduction into a target location of a body; and a second magnet assembly disposed opposite the first magnet assembly, wherein the first and second magnet assemblies are configured for applying a magnetic field to an operating region between the magnet assemblies.

Example 29: A magnetic navigation system for applying a magnetic field to an operating region within the body, the system comprising: first magnet assembly configured for introduction into a target location of a body; and a second magnet assembly disposed opposite the first magnet assembly, wherein the first and second magnet assemblies each comprise at least one magnet configured for applying a magnetic field to an operating region between the magnet assemblies.

Example 30: A magnetic navigation system for applying a magnetic field to an operating region within the body, the system comprising: a first magnet assembly configured for introduction into a target location of a body; and a second magnet assembly disposed opposite the first magnet assembly external to the body, wherein the first and second magnet assemblies each comprise at least one magnet configured for applying a magnetic field to an operating region between the magnet assemblies.

Example 31: A magnetic navigation system for applying a magnetic field to an operating region within the body, the system comprising: a first magnet assembly configured for introduction into a target location of a body; a second magnet assembly disposed opposite the first magnet assembly external to the body, wherein the first and second magnet assemblies are configured for applying a magnetic field to an operating region between the magnet assemblies; and at least one magnet assembly support for supporting at least one magnet assembly.

Example 32: A magnetic navigation system for applying a magnetic field to an operating region within the body, the system comprising: a first magnet assembly configured for introduction into a target location of a body; a second magnet assembly disposed opposite the first magnet assembly external to the body, wherein the first and second magnet assemblies are configured for applying a magnetic field to an operating region between the magnet assemblies; at least one magnet assembly support for supporting and moving at least one magnet assembly to change the direction of magnetic field applied to the operating region.

Example 33: A system for orienting a medical device within an operating region within the body, the system comprising: a first magnet assembly configured for introduction into a target location of a body; a second magnet assembly disposed opposite the first magnet assembly, wherein the first and second magnet assemblies are configured for applying a magnetic field to an operating region between the first and second magnet assemblies; a magnetically-responsive medical device.

Example 34: A magnetic navigation system for applying a magnetic field to an operating region within the body, the system comprising: an elongate body configured to be delivered within a body lumen, the elongate body including a distal portion and a proximal portion, wherein the distal portion comprises a first magnet assembly; a second magnet assembly disposed opposite the first magnet assembly external to the body, wherein the first and second magnet assemblies are configured for applying a magnetic field to an operating region between the first and second magnet assemblies.

Example 35: The system of any one of Examples 28-34, wherein the magnet assemblies including a positioner for rotating the magnet assemblies about a first axis and pivoting the magnet assemblies about a second axis.

Example 36: The system of any one of Examples 28-34, wherein the first and second magnet assemblies comprise at least one permanent magnet.

Example 37: The system of any one of Examples 28-34, wherein the first and second magnet assemblies comprise a compound permanent magnet comprising a plurality of segments of magnetic material.

Example 38: The system of any one of Examples 28-34, further comprising a positioner for rotating the magnet about a first axis and pivoting the magnet about a second axis.

Example 39: The system of any one of Examples 28-34, wherein the magnet assemblies being movably mounted on a support for movement about the body, and the magnet assemblies being positioned to project a magnetic field in a direction to orient a magnetically responsive medical device in a selected direction.

Example 40: The system of any one of Examples 28-34, wherein rotating and pivoting each magnet assembly to maintain the magnetic field direction projected by the magnet assemblies in relation to the magnetically-responsive medical device as the magnet assemblies move on the support about an operating region of the body to maintain the magnetically-responsive medical device in a selected orientation as the magnet assemblies move on the support.

Example 41: The system of any one of Examples 28-34, wherein the positions of the magnet assemblies are adjusted as the magnet assemblies move to change the direction of the magnetic field applied by the magnet assemblies to maintain the device in substantially the selected direction despite changes in the distance between the magnet assemblies and the operating region.

Example 42: The system of any one of Examples 28-34, further comprising a system controller to control a movement of a magnetically-responsive medical device.

Example 43: The system of any one of Examples 28-34, wherein controlling the positioners of each of the magnet assemblies to change the positions of the magnet assemblies in response to a user-input to apply the magnetic field in the operating region to cause the magnetically responsive device to orient substantially in the selected direction.

Example 44: The system of any one of Examples 28-34, wherein controlling the positioners of each of the magnet assemblies to change the positions of the magnet assemblies as the magnet assemblies move in order to maintain the magnetic field direction.

Example 45: The system of any one of Examples 28-34, wherein the controller controls the positioners in response to movement of the magnet assemblies, to apply a field whose direction is determined based upon a user-selected direction and a strength of the field in the operation region.

Example 46: The system of any one of Examples 28-34, wherein the first axes of the magnet assemblies are parallel. Each of the magnet assemblies may also comprise a positioner for controlling the position of the magnet, e.g. for rotating the magnet about a first axis and pivoting the magnet about a second axis, to selectively change the magnetic field applied by each magnet to the operating region in a body on the subject support. In a preferred embodiment, the magnet assemblies, and thus their respective magnets, are mounted on opposite sides of the operating region. The first axis of rotation of each magnet preferably extends through the respective magnet and the operating region, and preferably the first axes of rotation of the magnets are co-linear. The second axes of pivoting of each magnet is preferably perpendicular to the first axis, and also rotates about the first axis.

Example 47: The system of any one of Examples 28-34, wherein the first axes of the magnet assemblies are collinear and extend through the operating region.

Example 48: The system of any one of Examples 28-34, wherein the magnet assemblies each comprising a compound permanent magnet comprising a plurality of segments of magnetic material with differing magnetization directions so that relatively small rotations or pivots change the magnetic field projected by the magnet at a specific point.

Example 49: The system of any one of Examples 28-34, wherein the magnet assemblies are translatable along a first axis that extends radially outwardly from the center of the operating region, is pivotable about a second axis, generally perpendicular to the first axis that extends through the center of mass of the magnet, and is rotatable about the first axis.

Example 50: The system of any one of Examples 28-34, further comprising changing the magnetic moment of the magnetically-responsive medical device by selectively changing a physical condition of at least one magnet element in the magnetically-responsive medical device to change the orientation of the device with respect to the applied magnetic field.

Example 51: The system of any one of Examples 28-34, further comprising applying electrical energy to create temporary magnetic moments in one or more coils in the magnetically-responsive medical device to change the orientation of the magnetically-responsive medical device with respect to the static magnetic field, and orient the magnetically-responsive medical device in a selected direction within the operating region.

Example 52: A system for generating a magnetic field, the system comprising at least three magnetic field sources operable to radiate at least three magnetic fields into the anatomical body, where each magnetic field has a moment different from each moment of each of the other two magnetic fields relative to a fixed point in space.

Example 53: A magnetic field source for generating a magnetic field, said magnetic field source comprising a first coil corresponding to a first magnetic pole and a second coil corresponding to a second magnetic pole, wherein said first magnetic pole is moveable with respect to said second magnetic pole.

What is claimed is:

1. A magnetic navigation system for applying a magnetic field to an operating region within the body, the system comprising:
a first magnet assembly configured for introduction into a target location of a body; and
a second magnet assembly disposed opposite the first magnet assembly,
wherein the first and second magnet assemblies are configured for applying a magnetic field to an operating region between the magnet assemblies.

2. The system of claim 1, wherein the first and second magnet assemblies each comprise at least one magnet configured for applying the magnetic field to an operating region between the magnet assemblies.

3. The system of claim 1, wherein the second magnet assembly is disposed external to the body.

4. The system of claim 1, wherein the magnet assemblies including a positioner for rotating the magnet assemblies about a first axis and pivoting the magnet assemblies about a second axis.

5. The system of claim 1, wherein the first and second magnet assemblies comprise a single permanent magnet, a pair of permanent magnets, a single electromagnetic coil, two or more electromagnetic coils, or a combination thereof.

6. The system of claim 1, wherein the first and second magnet assemblies comprise a compound permanent magnet comprising a plurality of segments of magnetic material.

7. The system of claim 1, further comprising a positioner for rotating the magnet about a first axis and pivoting the magnet about a second axis.

8. The system of claim 1, wherein the magnet assemblies are movably mounted on a support for movement about the body, and the magnet assemblies being positioned to project a magnetic field in a direction to orient a magnetically responsive medical device in a selected direction.

9. The system of claim 1, wherein first axes of the magnet assemblies are parallel.

10. The system of claim 1, wherein first axes of the magnet assemblies are collinear and extend through the operating region.

11. The system of claim 1, wherein the magnet assemblies each comprise a compound permanent magnet comprising a plurality of segments of magnetic material with differing magnetization directions so that relatively small rotations or pivots change the magnetic field projected by the magnet at a specific point.

12. The system of claim 1, wherein the magnet assemblies are translatable along a first axis that extends radially outwardly from the center of the operating region, is pivotable about a second axis, generally perpendicular to the first axis that extends through the center of mass of the magnet, and is rotatable about the first axis.

13. A system for generating a magnetic field, the system comprising at least three magnetic field sources operable to radiate at least three magnetic fields into the body, wherein each magnetic field has a moment different from each moment of each of the other two magnetic fields relative to a fixed point in space.

14. A magnetic field source for generating a magnetic field, said magnetic field source comprising a first coil corresponding to a first magnetic pole and a second coil corresponding to a second magnetic pole, wherein said first magnetic pole is moveable with respect to said second magnetic pole.

15. A magnetic navigation system for applying a magnetic field to an operating region within the body, the system comprising:
a first magnet assembly configured for introduction into a target location of a body;
a second magnet assembly disposed opposite the first magnet assembly external to the body, wherein the first and second magnet assemblies are configured for applying a magnetic field to an operating region between the magnet assemblies; and
at least one magnet assembly support for one or more of supporting or moving at least one magnet assembly to change the direction of magnetic field applied to the operating region.

16. The system of claim 15, wherein the first and second magnet assemblies each comprise at least one magnet configured for applying the magnetic field to an operating region between the magnet assemblies.

17. The system of claim 15, wherein the second magnet assembly is disposed external to the body.

18. The system of claim 15, wherein the magnet assemblies including a positioner for rotating the magnet assemblies about a first axis and pivoting the magnet assemblies about a second axis.

19. The system of claim 15, wherein the first and second magnet assemblies comprise a single permanent magnet, a pair of permanent magnets, a single electromagnetic coil, two or more electromagnetic coils, or a combination thereof.

20. The system of claim 15, wherein the first and second magnet assemblies comprise a compound permanent magnet comprising a plurality of segments of magnetic material.

21. The system of claim 15, further comprising a positioner for rotating the magnet about a first axis and pivoting the magnet about a second axis.

22. The system of claim 15, wherein the magnet assemblies are movably mounted on a support for movement about the body, and the magnet assemblies being positioned to project a magnetic field in a direction to orient a magnetically responsive medical device in a selected direction.

23. The system of claim 15, wherein the positions of the magnet assemblies are adjusted as the magnet assemblies move to change the direction of the magnetic field applied by the magnet assemblies to maintain a device in substantially the selected direction despite changes in the distance between the magnet assemblies and the operating region.

24. The system of claim 15, wherein first axes of the magnet assemblies are parallel.

25. The system of claim 15, wherein first axes of the magnet assemblies are collinear and extend through the operating region.

26. The system of claim 15, wherein the magnet assemblies each comprise a compound permanent magnet comprising a plurality of segments of magnetic material with differing magnetization directions so that relatively small rotations or pivots change the magnetic field projected by the magnet at a specific point.

27. The system of claim 15, wherein the magnet assemblies are translatable along a first axis that extends radially outwardly from the center of the operating region, is pivotable about a second axis, generally perpendicular to the first axis that extends through the center of mass of the magnet, and is rotatable about the first axis.

28. A system for orienting a medical device within an operating region within the body, the system comprising:
a first magnet assembly configured for introduction into a target location of a body;
a second magnet assembly disposed opposite the first magnet assembly,
wherein the first and second magnet assemblies are configured for applying a magnetic field to an operating region between the first and second magnet assemblies; and
a magnetically-responsive medical device configured to magnetically interact with the first and second magnet assemblies.

29. The system of claim 28, wherein the first and second magnet assemblies each comprise at least one magnet configured for applying the magnetic field to an operating region between the magnet assemblies.

30. The system of claim 28, wherein the second magnet assembly is disposed external to the body.

31. The system of claim 28, wherein the magnet assemblies including a positioner for rotating the magnet assemblies about a first axis and pivoting the magnet assemblies about a second axis.

32. The system of claim 28, wherein the first and second magnet assemblies comprise a single permanent magnet, a pair of permanent magnets, a single electromagnetic coil, two or more electromagnetic coils, or a combination thereof.

33. The system of claim 28, wherein the first and second magnet assemblies comprise a compound permanent magnet comprising a plurality of segments of magnetic material.

34. The system of claim 28, further comprising a positioner for rotating the magnet about a first axis and pivoting the magnet about a second axis.

35. The system of claim 28, wherein the magnet assemblies are movably mounted on a support for movement about the body, and the magnet assemblies being positioned to project a magnetic field in a direction to orient a magnetically responsive medical device in a selected direction.

36. The system of claim 35, wherein rotating and pivoting each magnet assembly to maintain the magnetic field direction projected by the magnet assemblies in relation to the magnetically-responsive medical device as the magnet assemblies move on the support about an operating region of the body to maintain the magnetically-responsive medical device in a selected orientation as the magnet assemblies move on the support.

37. The system of claim 28, wherein the positions of the magnet assemblies are adjusted as the magnet assemblies move to change the direction of the magnetic field applied by the magnet assemblies to maintain the body in substantially the selected direction despite changes in the distance between the magnet assemblies and the operating region.

38. The system of claim 28, further comprising a system controller to control a movement of the magnetically-responsive medical device.

39. The system of claim 38, wherein the controller controls positioners of each of the magnet assemblies to change the positions of the magnet assemblies in response to a user-input to apply the magnetic field in the operating region to cause the magnetically responsive device to orient substantially in the selected direction.

40. The system of claim 38, wherein the controller controls positioners of each of the magnet assemblies to change the positions of the magnet assemblies as the magnet assemblies move in order to maintain the magnetic field direction.

41. The system of claim 38, wherein the controller controls positioners in response to movement of the magnet assemblies, to apply a field whose direction is determined based upon a user-selected direction and a strength of the field in the operation region.

42. The system of claim 28, wherein first axes of the magnet assemblies are parallel.

43. The system of claim 28, wherein first axes of the magnet assemblies are collinear and extend through the operating region.

44. The system of claim 28, wherein the magnet assemblies each comprise a compound permanent magnet comprising a plurality of segments of magnetic material with differing magnetization directions so that relatively small rotations or pivots change the magnetic field projected by the magnet at a specific point.

45. The system of claim 28, wherein the magnet assemblies are translatable along a first axis that extends radially outwardly from the center of the operating region, is pivotable about a second axis, generally perpendicular to the first axis that extends through the center of mass of the magnet, and is rotatable about the first axis.

46. The system of claim 28, further comprising changing the magnetic moment of the magnetically-responsive medical device by selectively changing a physical condition of at least one magnet element in the magnetically-responsive medical device to change the orientation of the body with respect to the applied magnetic field.

47. The system of claim 28, further comprising applying electrical energy to create temporary magnetic moments in one or more coils in the magnetically-responsive medical device to change the orientation of the magnetically-responsive medical device with respect to the static magnetic field, and orient the magnetically-responsive medical device in a selected direction within the operating region.

48. A magnetic navigation system for applying a magnetic field to an operating region within the body, the system comprising:
an elongate body configured to be delivered within a body lumen, the elongate body including a distal portion and a proximal portion, wherein the distal portion comprises a first magnet assembly;
a second magnet assembly disposed opposite the first magnet assembly external to the body, wherein the first and second magnet assemblies are configured for applying a magnetic field to an operating region between the first and second magnet assemblies.

49. The system of claim 48, wherein the magnet assemblies including a positioner for rotating the magnet assemblies about a first axis and pivoting the magnet assemblies about a second axis.

50. The system of claim 48, wherein the first and second magnet assemblies comprise a single permanent magnet, a pair of permanent magnets, a single electromagnetic coil, two or more electromagnetic coils, or a combination thereof.

51. The system of claim 48, wherein the first and second magnet assemblies comprise a compound permanent magnet comprising a plurality of segments of magnetic material.

52. The system of claim 48, further comprising a positioner for rotating the magnet about a first axis and pivoting the magnet about a second axis.

53. The system of claim 48, wherein the magnet assemblies are movably mounted on a support for movement about the body, and the magnet assemblies being positioned to project a magnetic field in a direction to orient a magnetically responsive medical device in a selected direction.

54. The system of claim 48, wherein the positions of the magnet assemblies are adjusted as the magnet assemblies move to change the direction of the magnetic field applied by the magnet assemblies to maintain the body in substantially the selected direction despite changes in the distance between the magnet assemblies and the operating region.

55. The system of claim 48, wherein first axes of the magnet assemblies are parallel.

56. The system of claim 48, wherein first axes of the magnet assemblies are collinear and extend through the operating region.

57. The system of claim 48, wherein the magnet assemblies each comprise a compound permanent magnet comprising a plurality of segments of magnetic material with differing magnetization directions so that relatively small rotations or pivots change the magnetic field projected by the magnet at a specific point.

58. The system of claim 48, wherein the magnet assemblies are translatable along a first axis that extends radially outwardly from the center of the operating region, is pivotable about a second axis, generally perpendicular to the first axis that extends through the center of mass of the magnet, and is rotatable about the first axis.
